(19) United States
(12) Patent Application Publication
(10) Pub. No.: US 2008/0288049 A1
(43) Pub. Date: Nov. 20, 2008

(54) Stent for In-Stent Restenosis

(75) Inventors: Eric J. Simso, Excelsior, MN (US); Matthew J. Miller, White Bear Lake, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/180,340

(22) Filed: Jul. 25, 2008

Related U.S. Application Data
(63) Continuation of application No. 09/681,118, filed on Jan. 12, 2001.

(51) Int. Cl. A61F 2/06

(52) U.S. Cl. 623/1.16

(57) ABSTRACT

A second stent having a wall thickness of 0.002 inches or less or a second stent having a tubular surface with openings therethrough where the ratio of the area of the surface to the area of the openings is at least 3:7 may be implanted in a first stent which has been previously implanted in a bodily vessel.

### CROSS-REFERENCE TO RELATED APPLICATIONS

This application is a Continuation of and claims priority to U.S. patent application Ser. No. 09/681,118, filed Jan. 12, 2001, the entire contents of which is hereby incorporated herein by reference.

### BACKGROUND OF INVENTION

A stenosis of an artery is a constriction or narrowing of the artery. The stenosis may be as a result of the buildup up of cholesterol, fat or other substances. Regardless of the cause of the stenosis, a stenosis can be life threatening. Stenoses of coronary arteries, for example, can diminish the flow of blood to the heart leading to heart damage and, possibly, death.

A number of methods have been developed for widening arteries which have become stenosed. These methods include stenting, percutaneous transluminal coronary angioplasty (PTCA), coronary artery bypass graft (CABG) procedures and the use of drugs and/or radiation.

Of the various methods of widening arteries which have become stenosed, stenting has proven to be of particular value in lowering the rate of restenosis or formation of new stenoses following the procedure. Even stented regions of a vessel, however, can restenose.

Restenosis only occurs in a fraction of stented patients. Estimates of restenosis rates range from roughly 15% of patients to as high as 30% to 40% for certain devices and vessels or lesions. It can, however, be difficult to treat in patients in whom it occurs. Restenosis typically occurs within less than six months of initial treatment of the vessel but may manifest itself many months to years later.

A number of different techniques have been devised to reduce the likelihood of restenosis in stented regions of a vessel. These techniques include treating the patient with anticoagulant and antiplatelet drugs and smooth muscle cell inhibitors. Direct delivery of drugs to the affected vessel is addressed in a plethora of patents including U.S. Pat. No. 5,861,168 which discloses the use of a stent bearing a nitric oxide precursor agent and U.S. Pat. No. 5,800,507 which discloses the use of a stent bearing fibrin. Other methods of treatment include delivery of radioactive substances to the affected region of the body as is disclosed in U.S. Pat. No. 5,871,437, U.S. Pat. No. 5,059,166 and U.S. Pat. No. 6,099,455. The use of ultrasonic energy in reducing the likelihood of restenosis is disclosed U.S. Pat. No. 5,836,896.

There remains a need for providing novel methods of dealing with restenosis in stented regions of a vessel as well as novel apparatuses for dealing with restenosis.

For the purpose of this disclosure, all U.S. patents and patent applications and all other publications referenced herein are incorporated herein by reference in their entirety.

A brief summary of the claimed embodiments of the invention is set forth below in accordance with 37 C.F.R. 1.73. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### SUMMARY OF INVENTION

In one embodiment, the invention is directed to a combination of a first stent which has been implanted in a bodily vessel and a second stent disposed within the first stent. At least a portion of the second stent has a wall thickness of 0.002 inches or less. Desirably, the entirety of the second stent has a wall thickness of 0.002 inches or less. More desirably, at least a portion of the wall thicknesses is 0.001 inches or less and most desirably, 0.0005 inches or less.

The first stent may be any suitable stent known in the art.

The second stent may be of any suitable design known in the art. Examples of stent designs include stents having one or more segments of interconnected struts and stents having one or more segments with cells and openings therethrough. Desirably, where the second stent has a tubular surface with openings, the second stent when expanded has a ratio of the area of the surface to the area of the openings of at least 3:10, desirably at least 1:2 and more desirably, at least 7.5:10. The second stent may be helical or non-helical.

The second stent may be made of any suitable material whether polymeric or metal or a combination of the two or otherwise. Where the second stent is made of foil, desirably the second stent is only one layer of foil thick. The second stent may be mechanically expandable or self-expanding. In the latter case, the second stent is desirably made of a shape memory material.

Optionally, the second stent may comprise a liner. The liner may be disposed within the second stent, outside the second stent or both inside and outside the second stent. The liner may be in the form of a membrane. Where a liner is provided, the liner may be made of any suitable material whether bioabsorbable or not. Suitable materials include graft materials. The liner may be made of polymeric materials and may be degradable or non-degradable, absorbable or non-absorbable. An example of a suitable non-bioabsorbable material is expanded PTFE (ePTFE). The bioabsorbable membrane or liner may further comprise a treatment agent.

In one embodiment, the second stent comprises at least a first support and a second support, and a liner extending between the first support and the second support. The liner may take the form of a metallic foil, a fabric or polymeric material such as Dacron or ePTFE, or a biomaterial such as collagen, elastin, autologous graft etc. Optionally, the first and second supports may be hoops.

The second stent may frictionally engage the first stent. Optionally, the second stent may be attached to the first stent, for example, via one or more hooks. The second stent may also be attached to the first stent by being bonded thereto. Examples of suitable adhesives for bonding the second stent to the first stent include fibrin adhesives. Other adhesives may also be used. Where the second stent is self-expanding, it may frictionally engage the first stent by self-expanding against the first stent and applying an outward force against the first stent. Hooks and/or adhesives may also be used to attach a self-expanding second stent to the first stent.

In another embodiment, the invention is directed to a combination of a first stent implanted in a bodily vessel and a second stent disposed within the first stent, where the second stent has a tubular surface with openings therethrough, and the ratio of the area of the surface to the area of the openings when the second stent is expanded is at least 3:10. More desirably, the ratio of the area of the surface to the area of the openings when the second stent is expanded is at least 1:2 and most desirably, the ratio is at least 7.5:10.

The invention is also directed to a method of stenting a previously stented region of a bodily vessel where the stented region of the bodily vessel has a first stent therein with a lumen therethrough. The method comprises the steps of disposing a second stent in the lumen of the first stent and implanting the second stent in the lumen of the first stent. At least a portion of the second stent has a wall thickness of 0.002 inches or less, more desirably, 0.001 inches or less, and most desirably, 0.0005 inches or less. The second stent may be of any suitable design as discussed above and may be mechanically expandable or self-expanding. The method may be used to treat a restenosed, previously stented area of a vessel or an area of stenosis just proximal or distal to the first stent.

In yet another embodiment, the invention is directed to a method of stenting a previously stented region of a bodily vessel. In accordance with the method, a second stent is disposed in the lumen of an already implanted first stent and implanted therein. The second stent has a tubular surface with openings therethrough. When the second stent is expanded, the ratio of the area of the surface to the area of the openings is at least 3:10, more desirably 1:2 and most desirably, 7.5:10. The method maybe used to treat a restenosed, previously stented area of a vessel.

In any of the inventive combinations and methods disclosed herein, the entirety of the second stent may be disposed within the first stent or at least a portion of the second stent may extend outward from at least one of the first and second ends of the first stent.

The inventive methods may be carried out at any point subsequent to implantation of the first stent using any suitable first stent and second stent as discussed above. Desirably, the first stent will have been implanted in the bodily vessel for a period of time up to one month and more desirably, for a period of time between one month and six months. Even more desirably, the first stent will have been implanted for a period of time in excess of one half year prior to insertion of the second stent. Most desirably, the first stent will have been implanted in the bodily vessel for a period of time in excess of one year.

The inventive methods may be preceded and/or followed by one or more steps in which the stented region of the vessel is treated to reduce restenosis.

A detailed description of the invention in its various embodiments is provided below.

### BRIEF DESCRIPTION OF DRAWINGS

FIG. 1 shows a perspective view of the inventive stent combination in a vessel with parts cut away.

FIG. 2 shows a cross-section of the inventive combination of FIG. 1 taken along line 2-2.

FIG. 3 shows a perspective view of a stent.

FIG. 4 shows a perspective view of a stent.

FIG. 5 shows a stent with a membrane.

FIG. 6 shows a stent having a plurality of supports and a liner.

FIG. 7 shows a first stent and a second stent with hooks.

FIG. 8 shows a second stent extending from the end of a first stent.

### DETAILED DESCRIPTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, unless otherwise indicated, like reference numerals in the figures shall refer to like structures.

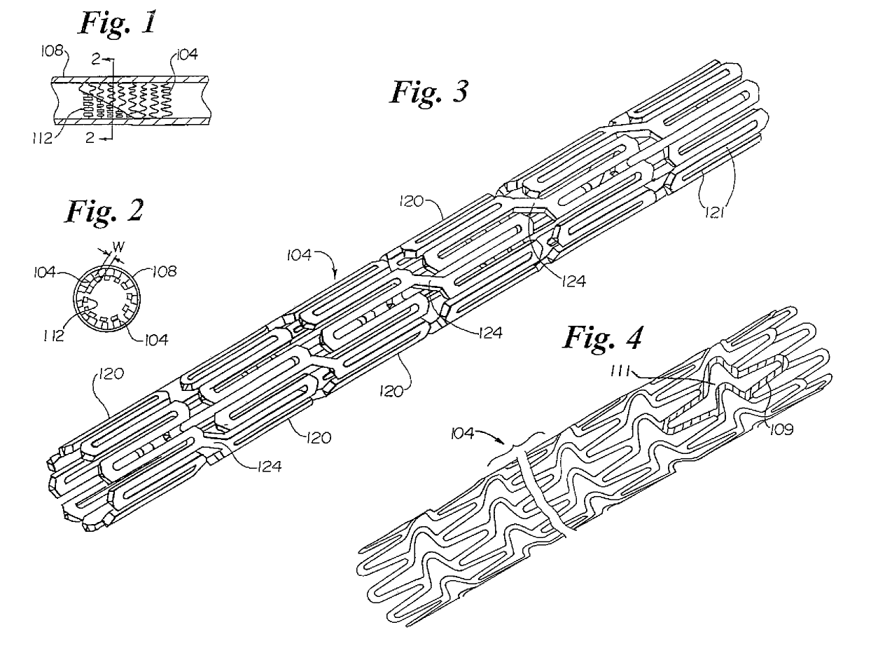

Turning to FIGS. 1 and 2, the invention is directed, in one embodiment, to a combination of a first stent 104 implanted in a bodily vessel 108 and a second stent 112 disposed within first stent 104 where at least a portion of second stent 112 has a wall thickness W of 0.002 inches or less and desirably, 0.001 inches or less. Even more desirably, the wall thickness is 0.0005 inches or less and most desirably, the wall thickness is 0.00025 inches or less.

First stent 104 may be any suitable stent as known in the art for implantation in a bodily vessel. Desirably, the stent comprises one or more serpentine segments 120 as shown in FIG. 3. The stent of FIG. 3 comprises a plurality of segments 120 joined by angled connectors 124. Each serpentine segment comprises a plurality of connected struts 121. Adjacent serpentine segments 120 are joined by connectors 124. Optionally, connectors 124 have one or more bends therein. The ends of the connectors may be circumferentially displaced as shown in FIG. 3 or circumferentially aligned with one another. Another example of a suitable stent is shown in FIG. 4. Stent 104, as shown in FIG. 4, includes a plurality of interconnected closed cells 109 (one of which is shown shaded). Each closed cell 109 includes an opening 111 therethrough. Stents having at least one segment with closed cells therein are also disclosed in U.S. Pat. No. 5,733,303, U.S. Pat. No. 6,059,810 and U.S. Pat. No. 6,033,433. Other examples of first stents include those disclosed in commonly assigned, copending U.S. application Ser. No. 09/437,049, spiral or helical stents, stents having a constant diameter when expanded, stents at least a portion of which taper whether an end portion, a middle portion or any other portion and conical stents. The first stent may include one or more radiopaque portions such as is disclosed in commonly assigned copending U.S. application Ser. No. 09/659,571.

First stent 104 may be mechanically expandable, for example balloon expandable or may be self-expandable.

The first stent may be made of any suitable metallic material or combination of metallic materials including tungsten, aluminum, metal alloys, stainless steel, tantalum, rhenium, titanium, or other types of metallic materials. Alternatively, non-metallic materials, composites of metallic and non-metallic materials or other composites may be used. Metal-plastic or metal-ceramic composites may be used. Non-metallic materials such as rubbers, ceramics, plastics or other polymers may also be used. The stent may be optionally made of shape memory materials, whether metal, polymeric or otherwise.

Second stent 112 desirably is self-expandable and may be made of any suitable material including those specifically disclosed above. Desirable shape memory materials include Nitinol as disclosed in WO 96/26689 and U.S. Pat. No. 6,059,810. The invention also contemplates the use of second stents which are mechanically expandable, for example, by balloon. An example of such a stent is disclosed in U.S. Pat. No. 5,733,303. Second stent 112 may also be a foil stent. Where a foil stent is used, the foil stent is desirably only one layer of foil thick. More information about foil stents may be found in U.S. Pat. No. 6,120,535.

Second stent 112 may be of any suitable design including the designs discussed above for the first stent. Desirably, the stent comprises one or more serpentine segments such as those shown at 120 in FIG. 3. Adjacent segments may be connected via angled connectors as shown at 124 in FIG. 3 or non-angled connectors. As with the first stent, the second stent may also be formed of a plurality of serpentine segments joined by connectors having one or more bends therein. The ends of the connectors may be circumferentially aligned with one another or circumferentially displaced from one another. The second stent may include a plurality of interconnected closed cells such as those shown at 109 in FIG. 4. Stents having at least one segment having closed cells therein are also disclosed in U.S. Pat. No. 5,733,303, U.S. Pat. No. 6,059,810 and U.S. Pat. No. 6,033,433.

Where the stent comprises a plurality of struts, such as that shown in FIGS. 3 and 4, the struts are desirably of a thickness of 0.001 inches or less, more desirably 0.0005 inches or less and, most desirably, 0.00025 inches or less. Such stents may desirably be provided in a foil stent as discussed above.

Another example of a suitable second stent is disclosed in commonly assigned, copending U.S. application Ser. No. 09/437,049 which discloses stents having a microstructure which preferably have about the following dimensions:
strut width 0.00025-0.002 inches
strut thickness 0.00025-0.004 inches
maximum PIN opening 0.002-0.020 inches diameter.

The term “maximum PIN opening” is used herein to describe micro openings in which the dimensions specify the largest pin which can be passed through the cell opening. This applies as noted above to the expanded stent configuration. Typically, as a stent is expanded to larger diameters, the opening becomes larger. It is believed that using a maximum PIN opening specification that the concept of the present invention may be more readily applicable to stents of either open or closed cell geometries.

Other examples of a second stent include spiral or helical stents, stents having a constant diameter when expanded and stents at least a portion of which taper whether an end portion, a middle portion or any other portion and conical stents.

The second stent may include one or more radiopaque portions such as is disclosed in commonly assigned copending U.S. application Ser. No. 09/659,571. The radiopaque portions of the second stent when expanded may line up with one or more radiopaque portions of the first stent or may be displaced from any radiopaque portions of the first stent.

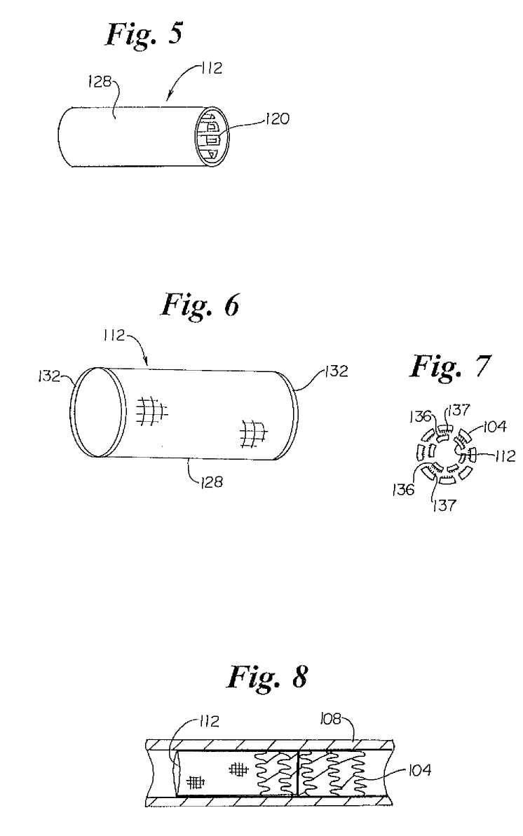

As shown in FIG. 5, second stent 112 may comprise an optional liner 128 disposed on the outer surface of the stent or stent segments 120. The invention also contemplates the presence of an optional liner in the interior of the stent or a sandwich construction, for example ePTFE-stent-ePTFE. Liner 128 may be any suitable graft material including expanded PTFE (ePTFE) or Dacron. Liner 128 may also be a bioabsorbable membrane including, for example, ethylene vinyl acetate (EVA), or poly (D,L-lactide) PLA. Other suitable materials for liner 128 include foils, synthetic polymeric materials including polyvinylpropylene (PVP), polyacrylamide (PA), and biomaterials such as collagen and elastin.

Second stent 112 may also comprise a plurality of supports 132 with a liner 128 extending therebetween, as shown in FIG. 6. Desirably, supports 132 are in the form of hoops. Liner 128 has a thickness of less than 0.002 inches and more desirably, less than 0.001 inches. Even more desirably, the thickness is 0.0005 inches or less and most desirably, the thickness is 0.00025 inches or less. Liner 128 may be made of foil or any suitable graft material including bioabsorbable materials and ePTFE.

The second stent may optionally be attached to the first stent. The attachment may be through one or more hooks which may be secured to one of the first or second stents. Optionally, the other of the first or second stents may comprise a plurality of loops for receiving the hooks. Desirably, the hooks and loops operate similar to VELCRO™. As shown in FIG. 7, hooks 136 extending from second stent 112 engage loops 137 extending from first stent 104. The second stent may also be bonded to the first stent via the use of adhesives.

The second stent may apply an outward force against the first stent and form a friction fit with the first stent as is the case with a self-expanding stent.

Where the second stent has a tubular surface with openings therethrough, as shown for example in FIG. 4, desirably, the ratio of the area of the surface to the area of the openings at least 3:10 when the second stent is expanded. More desirably, the ratio of the area of the surface to the area of the openings when the second stent is expanded is at least 1:2. Even more desirably, the ratio of the area of the surface to the area of the openings is at least 7.5:10. Providing a high ratio of surface area to the area of the openings is desirable in preventing additional restenosis.

The second stent may be longer or shorter than the first stent or may be the same length as the first stent. As such, second stent 112 may be entirely contained within first stent 10 as shown in FIG. 1 or may extend outward from one or both ends of the first stent. As shown in FIG. 8, second stent 112 extends outward from one end of first stent 104. Desirably, the overlap between the first and second stents will be anywhere from 10% to 100% and more desirably from 50% to 100% where the percentage overlap is calculated based on the ratio of the length of the region of overlap to the length spanned from the first end of the combination of the first and second stents to the second end of the combination of the first and second stents. Thus, where one stent is totally contained within the other stent, or where one stent extends from both ends of the other stent, the length of the combination of the first stent and second stent is equal to the length of the longer of the two stents. Where one stent extends from only one of the ends of another stent, the length of the combination of the first stent and second stent is equal to the length of the two stents minus the length of the region of overlap. By allowing the second stent to extend from one or both ends of the first stent, any stenosed or restenosed areas adjacent to one or both ends of the first stent may also be stented. This may prove particularly beneficial in cases where restenosis has occurred at one or both ends of an implanted step because of edge effects.

The invention is also directed to the combination of a first stent implanted in a bodily vessel and a second stent disposed within the first stent, as shown, for example, in FIG. 1, where the second stent has a tubular surface with openings therethrough. When the second stent is expanded, the ratio of the area of the surface to the area of the openings at least 3:10. More desirably, the ratio of the area of the surface to the area of the openings is at least 1:2. Even more desirably, the ratio of the area of the surface to the area of the openings is at least 7.5:10.

Desirably, in any of the above embodiments, the second stent comprises a treatment agent. The treatment agent may be applied to any portion or the entirety of the stent. In one embodiment, where the second stent comprises a liner, the treatment agent may be applied to the liner. The liner may be in the form of a polymer that degrades or in the form of a polymer that persists as the treatment agent is released. In another embodiment, where the second stent comprises a plurality of struts and/or segments, the treatment agent may be applied to at least a portion of the struts and/or segments.

Suitable treatment agents include hydrophilic drugs such as heparin or hirudin to protect against coagulation. Hydrophobic drugs such as prostaglandins or aspirin and vitamin E may be used to protect against platelet activation. Vitamin E and other anti-oxidants such as sodium ascorbate, phendies, carbazoles, and tocotrienols may be used to protect against oxidation. The treatment agent may also include prostaglandins PGI2 or PGE2, RGD peptide, thrombomodulin, TPA (Tissue Plasminogen Activator) and Urokinase as well as other bioactive proteins. Gene therapy agents such as antiplatelet and antibody fragments, for example GB2B3A may also be provided. Other suitable agents include nitric oxide precursor agents, fibrin, Taxol and ticlopidine. Any of the treatment agents disclosed in U.S. Pat. No. 6,074,659 U.S. Pat. No. 6,120,847, U.S. Pat. No. 5,861,168, U.S. Pat. No. 5,800,507 and U.S. Pat. No. 5,693,085 and any of the methodologies disclosed therein for delivering treatment agents may be used. More generally, the treatment materials may include the known antithrombic agents, anti-proliferative agents, antibacterial and/or antimicrobial agents and antifungal agents.

The treatment agent may also be radioactive. The radioisotope used in the treatment agent may be an alpha, beta or gamma emitter. Desirably, the half-life of the radioisotope is between 10 hours and 100 days. More desirably, the radioisotope will be a beta emitting isotope such as phosphorous 32, with a 14.3 day half-life and no gamma rays. Additional details concerning such a treatment agent may be found in U.S. Pat. No. 5,722,984 and U.S. Pat. No. 5,871,437. Other suitable radioisotopes for use as treatment agents include the beta emitting isotope gold 198 (half-life 2.7 days) as disclosed in U.S. Pat. No. 5,059,166, beta emitting strontium with a half-life of approximately 50.5 days as disclosed in U.S. Pat. No. 6,129,658, sulfur as disclosed in U.S. Pat. No. 5,919,126 and beta particle radioactive metals selected from the group consisting of rhenium, copper, dysprosium, yttrium, holmium, praseodymium, lanthanum, samarium, gold, and combinations thereof as disclosed in U.S. Pat. No. 6,077,413.

Where the treatment agent comprises a radioisotope, any suitable method of applying the radioisotope, including those disclosed in the above-mentioned patents, may be used.

In yet another embodiment, the invention is directed to a method of treating restenosis in a stented region of a bodily vessel where the stented region of the bodily vessel has a first stent therein with a lumen therethrough. The method comprises the steps of disposing a second stent in the lumen of the first stent and implanting the second stent in the lumen of the first stent. At least a portion of the second stent has a wall thickness of 0.002 inches or less and more desirably of 0.001 inches or less. Even more desirably, the wall thickness is 0.0005 inches or less and most desirably, the wall thickness is 0.00025 inches or less.

Desirably, where the second stent has a tubular surface with openings therethrough, the ratio of the area of the surface to the area of the openings when the second stent is expanded is at least 3:10, more desirably, at least 1:2 and most desirably at least 7.5:10.

In another embodiment, the invention is directed to a method of treating restenosis in a stented region of a bodily vessel, the stented region of the bodily vessel having a first stent therein with a lumen therethrough, the method comprising the steps of disposing and implanting a second stent in the lumen of the first stent. The second stent has a tubular surface with openings therethrough. The ratio of the area of the surface to the area of the openings when the second stent is expanded is at least 3:10, desirably, at least 1:2 and most desirably at least 7.5:10.

Any of the inventive methods disclosed above may employ any of the second stents disclosed above, whether self-expanding, mechanically expandable or otherwise expandable.

The inventive methods of implanting a second stent within an already implanted first stent may be practiced after the first stent has been implanted for at least a month, two months, six months, a year or more. More generally, the second stent may be implanted at any time subsequent to the deployment of the first stent. Typically, the method will be carried out subsequent to restenosis of the stented region of the vessel.

Any of the inventive methods disclosed herein may also include one or more treatment steps in which the stented region of the vessel is treated to reduce or eliminate restenosis, desirably, prior to insertion of the second stent in the first stent. Although specific treatment processes are discussed briefly below, any suitable treatment process for reducing or eliminating restenosis may be used.

Balloon angioplasty, a technique in which a balloon is expanded in a stenotic region of a vessel, may be employed. Additional details concerning balloon angioplasty may be found in U.S. Pat. No. 6,010,480. Another technique which may be employed involves the use of a thermal catheter comprising a balloon as disclosed in U.S. Pat. No. 4,799,479. Yet another technique which may be used employs both a balloon and a laser as disclosed in U.S. Pat. No. 5,741,246. Other suitable techniques for reducing restenosis involve the use of a laser catheter as disclosed in U.S. Pat. No. 6,106,51. The laser may be an excimer laser, Nd:YAG, holmium, CO.sub.2 laser or any other suitable laser.

Techniques in which plaque is mechanically removed from the stented region may also be employed. U.S. Pat. No. 5,941,869, for example, describes a debulking process which employs a catheter system having a stenotic material removal mechanism mounted on a distal portion of an elongated inner catheter. Rotational atherectomy is yet another technique that may be used. Rotational atherectomy incorporates a rotary file or burr into the distal portion of a catheter to remove plaque. Transluminal extraction catheter atherectomy involves the use of a catheter with tip-mounted cutting blades to remove plaque.

Another treatment modality involves the use of radiation treatment, as disclosed in U.S. Pat. No. 5,833,593.

Yet another approach involves treatment with a drug which modulates cell growth into the target artery to inhibit the proliferation of smooth muscle cells. In particular, antiplatelet agents such as aspirin and dipyridamole, and anticoagulants such as heparin, have inhibited platelet aggregation and thrombus formation to a limited degree, thereby reducing the risk of early occlusion. Other suitable treatment agents are disclosed above.

The inventive combinations and methods may be used for coronary arteries, peripheral arteries, arteries of the neck and intracranial arteries. More generally, the inventive combinations and methods may be used for any vessel of the human body including but not limited to arteries, veins, biliary ducts, urethras, fallopian tubes, bronchial tubes, the trachea and the esophagus.

In addition to the specific embodiments claimed below, the invention is also directed to other embodiments having any other possible combination of the dependent features claimed below in conjunction with the independent claim from which they depend. To that end, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below (e.g. claim 3 may be taken as alternatively dependent from claim 2; claim 4 may be taken as alternatively dependent on claim 2, or on claim 3; claim 6 may be taken as alternatively dependent from claim 5; etc.).

The above disclosure is intended to be illustrative and not exhaustive. This description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is as follows:

1. In combination, a first stent and a second stent at least partially disposed within the first stent, wherein at least one of said stents is a foil stent.

2. The combination of claim 1, wherein each of said stents is a foil stent.

3. The combination of claim 2, wherein a foil surface of the first stent contacts a foil surface of the second stent.

4. The combination of claim 1, wherein the first stent is attached to the second stent via a hook and loop fastening system.

5. The combination of claim 1, wherein the second stent has a wall thickness of 0.00025 inches or less.

6. The combination of claim 1, wherein the second stent consists of a foil.

7. The combination of claim 6, wherein the second stent is only one layer of foil thick.

8. The combination of claim 6, wherein the first stent consists of a foil.

9. The combination of claim 8, wherein the first stent is only one layer of foil thick

10. The combination of claim 8, wherein a foil surface of the first stent contacts a foil surface of the second stent.

11. The combination of claim 1, wherein the combination does not comprise a graft.

12. A combination comprising:
a first stent consisting of an expandable framework; and
a second stent at least partially disposed within the first stent, the second stent consisting of foil.

13. The combination of claim 12, further comprising a hook and loop fastening system, wherein the first stent is attached to the second stent via the hook and loop fastening system.
